(19) United States
(12) Patent Application Publication
(10) Pub. No.: US 2014/0107740 A1
(43) Pub. Date: Apr. 17, 2014

(54) Advanced Electrolytic Device--Bimetallic Wound Dressing

(71) Applicants: Anita Crisp, Prescott, AZ (US); enVITA II, LLC, Prescott, AZ (US)

(72) Inventor: William E. Crisp, Paradise Valley, AZ (US)

(73) Assignee: enVITA II, LLC, Prescott, AZ (US)

(21) Appl. No.: 13/833,393

(22) Filed: Mar. 15, 2013

Related U.S. Application Data
(63) Continuation-in-part of application No. 13/539,968, filed on Jul. 2, 2012.
Application No. 13/539,968 is a division of application No. 12/380,111, filed on Feb. 23, 2009, now abandoned.
Application No. 12/380,111 is a continuation-in-part of application No. 11/130,800, filed on May 17, 2005, now Pat. No. 7,495,146.
Application No. 11/130,800 is a continuation-in-part of application No. 10/891,566, filed on Jul. 15, 2004, now abandoned.

(51) Int. Cl. A61N 1/04

(52) U.S. Cl. CPC A61N 1/0468
USPC 607/115

(57) ABSTRACT

An Advanced Electrolytic Device-Bimetallic Wound Dressing that provides galvanic current and has antimicrobial properties. The dressing comprises a pliable base material, along with a pattern of deposited metal powder and flakes. The metal powder and flakes include at least two different metal types. The two different metals create a galvanic current in the wound area.

### CROSS REFERENCE TO RELATED APPLICATIONS

This patent application is a continuation-in-part of U.S. Ser. No. 13/539,968 filed 2 Jul. 2012, which was a divisional of U.S. application Ser. No. 12/380,111 filed 23 Feb. 2009, which was a continuation-in-part of U.S. application Ser. No. 11/130,800, filed May 17, 2005, which was a continuation-in-part of U.S. application Ser. No. 10/891,566, filed Jul. 15, 2004, each of which is hereby incorporated herein by reference.

### BACKGROUND OF THE INVENTION

1. Field of the Invention

The present invention relates to an apparatus for attacking microbes, namely, bacteria, viruses, and fungi. More particularly, the present invention is a sustained release galvanic current bandage or gauze for use as a wound dressing.

2. Description of Related Art

Silver has been used as a disinfectant for centuries. The use of medicinal silver was diminished by the advent of more versatile and effective antibiotics. The misuse of antibiotics coupled with bacteria's ability to mutate have resulted in resistant organisms and reawakened interest in silver's effective antimicrobial properties. Elemental silver is an effective microbicide in solutions as dilute as one part per 100 million. Silver ions kill micro-organisms by blocking the respiratory system, which is the process of harvesting energy by transferring electrons from an electron donor to an electron receptor.

Although salts of silver will immediately supply bactericidal qualities of silver to a wound, the salts also can impair wound healing. Ionic silver decreases the inflammatory process in a wound, decreases edema, and increases blood supply to the wound. Silver alone decreases wound surface zinc, which is required for metalloproteinase (MMP) activity modulation. Silver and zinc together also increase wound calcium which increases the wound re-epithelization rate. Matrix MMPs are a group of proteolytic enzymes that are vital in various modeling repairs and the inflammatory processes of wound healing. There are now 20 MMPs identified. MMPs are dependent on intrinsic zinc ions and extrinsic calcium for full activity in modifying the inflammatory response by binding histidine. MMPs are produced by a number of important cells vital to wound repair. For example, neutrophils, macrophages, keratinocytes, and fibroblasts are expressed in physiologic repair, remodeling and epithelial proliferation in wounds.

[U.S. Pat. No. 5,298,017 to Theeuwes et al. (the '017 patent), which is incorporated herein by reference, describes an iontophoretic process by which drugs are delivered transdermally or transmucosally under the influence of an electrical potential. Iontophoretic devices use two distinct electrodes, with at least one of the electrodes being applied to the body. These devices typically utilize a conventional electric power source, such as a battery, to develop the electric current. In some cases, the power source is located separately from the device and in some cases the power source is integrated into the device. These devices also rely solely on the creation of a discrete ion pathway incorporating the body or tissue to effect an electromotive force via forms defined by the sequence of a first electrode, tissue and a second electrode.

There are devices described in the prior art that rely on the electric field generated by the device itself The power source generally provides no therapeutic value itself other than to provide the electric current necessary to drive the iontophoretic or electro-osmotic device to deliver an agent that is different from the electrode metals. Further, if the power supply should fail for any reason, the device is typically rendered useless. Also, where the power source is located away from the device, limitations are imposed on patient mobility. Still further, even when the prior art integrates the conventional power source into the device, there are limitations. In particular, the prior art makes it clear that the conventional power source must be protected from short circuiting itself Consequently, great lengths have been taken to insure that the two electrodes are insulated in order to limit the possibility of a short circuit. Further limitations of these devices include high cost due to wires, electrical insulation, battery failure, problems with user compliance, maintenance, and damage.

In spite of the fact that the use of external power sources is prevalent in the art of iontophoresis and electro-osmosis, it is known to rely exclusively on the electric potential generated by the galvanic couple between dissimilar materials, e.g., a zinc electrode and a silver/silver chloride counter electrode, to deliver a drug. For example, the embodiment of the device illustrated in FIG. 2 of the '017 patent does not use an external power source. While the primary purpose of such devices is to deliver a drug present in a drug reservoir, as a consequence of the galvanic couple, ions of the materials used for the anode and/or cathode are delivered into the body. Unfortunately, because the anode and cathodes of such prior art devices are typically made from materials having a relatively low total surface area, the rate of metallic ion transfer from the metallic electrodes is typically lower than desired for satisfactory therapeutic effects.

As described in U.S. Pat. No. 5,814,094 to Becker et al. (the '094 patent), iontophoretic devices that provide silver ions for wound healing are known. Use of silver-coated nylon as the anode for the iontophoretic device of the device of the '094 patent provides a relatively high total surface area material as the source of silver ions. However, the device of the '094 patent features the use of an external power source connected to the silver-coated nylon anode to generate the electrical potential that drives the silver ions into the body, and so suffers from the limitations of other iontophoretic devices described above.

U.S. Pat. No. 6,522,918 to Crisp et al. (the '918 patent), which is incorporated herein by reference, describes electrolytic devices for use in treating tissue through the use of a silver-bearing material and a metal other than silver with no external voltage source necessary. However, one of the disadvantages of the devices of the '918 patent is that the devices are limited in usefulness due to their inherent short-lived duration of action. U.S. Pat. No. 7,495,146, also incorporated herein by reference by virtue of the present application's being a continuation in part of both it and its lineage of prior provisional and subsequent Continuation-in-Part applications, discloses a wound dressing material containing silver and zinc in which the metal particles enjoy sustained delivery, giving a prolonged galvanic current, due in part to the erosion of a polymer used to create the wound dressing. While useful, the dressings of U.S. Pat. No. 7,495,146 do provide a diffuse galvanic current to the wound but a more aggressive and concentrated galvanic current in the wound site would create an even better environment for wound healing, and the present invention provides this improvement.

Therefore, a need exists for a wound dressing bandage of permeable material fabric or gauze that provides a concerted galvanic current for extended periods of time in the area of a wound covered by the present wound dressing as disclosed and claimed herein.

### SUMMARY OF THE INVENTION

The present invention is directed to a galvanic current wound dressing device having antimicrobial properties and to a method for treating a patient with the wound dressing. The dressing comprises a pliable base material, and a pattern of deposited medical grade silver metal powder and medical grade zinc metal flakes in a pattern whereby the metals comprise at least two metals which, upon nearly touching, generate a more efficient and sustainable galvanic current. The layout is such that the ratio of metallic formulations is as close to one-to-one silver to zinc as possible. Currently, this is done via screening 2 mm and 1 mm circles in an equal layout such that each silver dot is surrounded by at least 4 half dots of zinc to get the desired ratio of metals, all applied to the same side of said fabric. Spacing between metal formulations is approx. 1 mm in alternating rows. This can be done via many printing applications such as but not limited to ink-jet/blotter printing, silk screening, rotogravure, etc., Note that the actual geometry of the “pattern” is not material to patentability. What is important is the ratio of the two different metals and the spacing that is in between the two. Our studies have shown that the closer the metals are to each other, without touching, the more current potential exists due to the lower resistance between the two. Each different type of metal will have an optimum spacing as it relates to silver. In addition the silver mixture can be applied on one side of the fabric/permeable surface and the zinc mixture can be applied on the other side of the fabric/permeable surface; as long as the material is water permeable, this latter layout works as well as the single surface area application without weakening the galvanic current needed for wound healing.

In addition, the investigation of this invention has shown that the two metals can be touching and/or overlapping IF a third printing process is added to provide a non-conductive water soluble carrier/binder printed between the metals, to prevent a short in the current.

### BRIEF DESCRIPTION OF THE DRAWING(S)

FIG. 1: FIG. 1 is a plan view of a section of a wound dressing according to the present invention, showing 43 columns and 43 rows of arranged deposited metals;

FIG. 2: FIG. 2 is a plan view of a section of a wound dressing according to the present invention, in which each silver dot is surrounded by at least 4 half dots of zinc to get the desired ratio of metals;

FIG. 3: FIG. 3 is a diagram of a deposition pattern which is alternative to the deposition pattern shown in FIG. 1 and

FIG. 4: FIG. 4 is a diagram of a further deposition pattern which is alternative to the deposition pattern shown in FIG. 1.

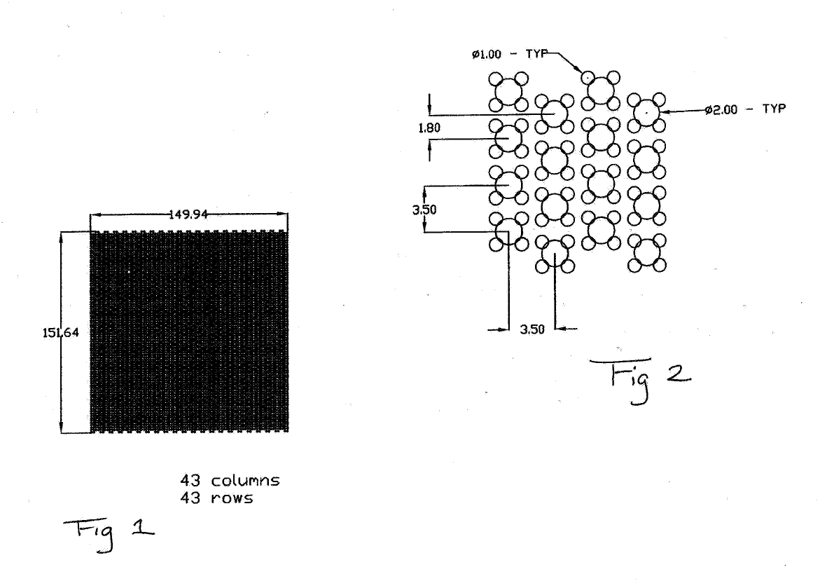

### DETAILED DESCRIPTION OF THE INVENTION

The present application is directed to a galvanic current wound dressing having antimicrobial properties and to a method for treating a patient with the wound dressing. The dressing comprises a pliable base material, and a pattern of deposited medical grade silver metal powder and medical grade zinc metal flakes in a pattern whereby the metals comprise at least two metals which, in close proximity but not touching, generate a galvanic current and which patterns of deposited metals are present in a repeating pattern. The deposited medical grade silver metal powder circles and medical grade zinc metal flakes are adhered to the pliable base material by a non-conductive water soluble carrier/binder. The carrier/binder (binding substance) binds the metals in suspension and is of a water soluble, solid formulation. This binder, as used, is non-conductive in function. As the binder reduces in mass, additional metallic materials contact the activation ingredient (saline) allowing for additional ionic activity between the dissimilar metals. The “pattern” is not relevant. What is important is the ratio of the two different metals. The realistic minimum ratio for application is 15% metal, 85% binder. The ratio of the 2 metallic formulations to each other is one-to-one. This is for ease of manufacturing and in practice may vary by about 10% either way.

The pattern of deposition in small and large circles is representative only—the silver and zinc in their respective carrier solutions may be deposited in any repeating pattern. Metals other than silver and zinc may be used—any two metals which spur the formation of a galvanic current may be used in the practice of the present invention. Strictly speaking, any metal types can be used in which the first metal particles have an electrochemical potential and the second metal particles have a different electrochemical potential from the plurality of first metal particles when the first metal is silver or a salt or oxide of silver, the second metal can include, without limitation, pure or nearly pure aluminum, cobalt, copper, gold, iron, magnesium, platinum, titanium or zinc, and/or suitable salts and oxides thereof.

As explained above, in addition two metals can be touching and/or overlapping IF a third printing process is added so that there is a non-conductive water soluble carrier/binder printed between the metals to prevent a short in the circuit.

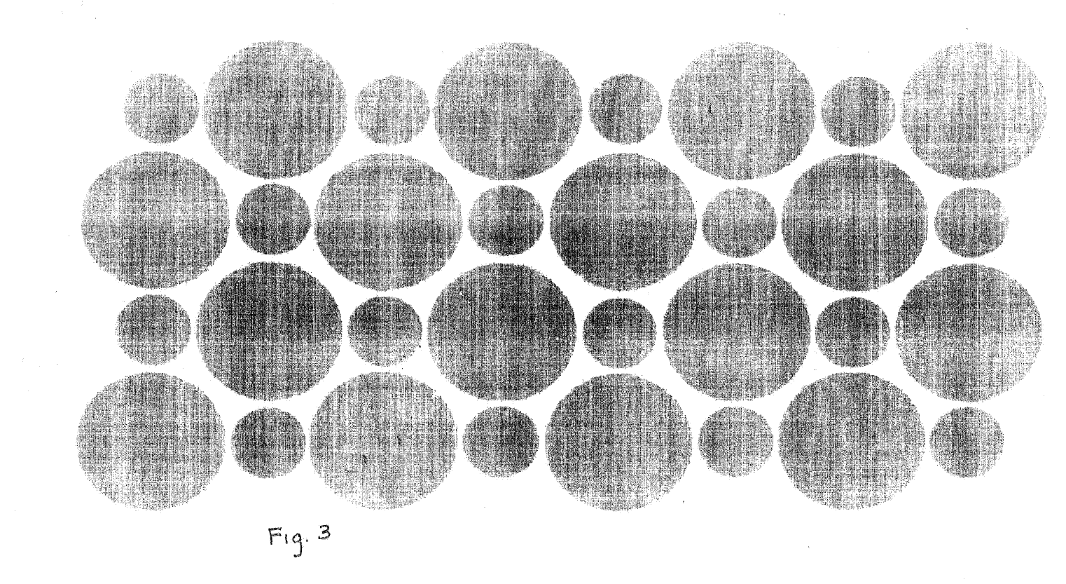

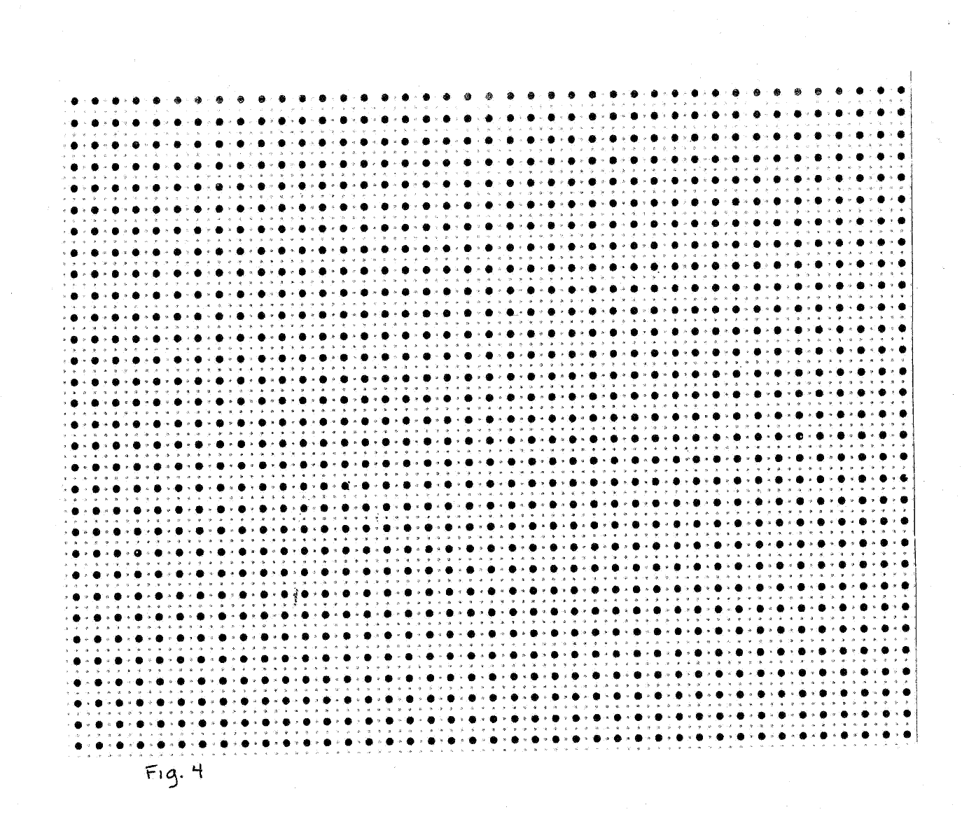

It should be noted that the metal described herein must be applied to a pliable base, typically a medical grade 100% polyester fabric material; however, other materials can be used such as gauze or open-celled foam or other materials as long as they are pliable. Typically non-allergenic materials, compatible to the skin or wound bed, are used so as not to irritate the skin or wound bed. Onto the pliable base material are deposited metal particles in a carrier/binder in which the first metal particle type is deposited in a pattern of larger circles, and flakes of the second metal particle type are deposited in a pattern of smaller circles. The larger and smaller circles can be spaced as shown in FIGS. 3 and 4 (close, not touching) or overlapping in plan view, as long as a third layer of a non-conductive water soluble carrier/binder is present between the otherwise overlapping patterns (FIG. 2). The circles of FIG. 2 thus make up of a combination of the applicable metals as described with the a non-conductive water soluble carrier/binder which binds the metals in suspension and is of a water soluble, solid formulation.

The silver particles of the present invention are preferably silver that is at least 99.99% to 99.9999% pure. However, less pure silver, and suitable salts and oxides thereof may be used but not as effectively. Examples of other silver particles that may be used include: silver fluorides, silver chlorides, silver bromides, silver iodides, silver oxides, silver sulfides, silver selenides, and silver tellurides.

The embodiment of the present invention, a wound dressing bandage, may be made any shape or size. For example, the wound dressing bandage may be spherical, triangular, rectangular, etc. It may also be designed to fit a particular area of the body. For example, it can be larger in size to provide localized treatment to the back areas of a patient, or smaller in size to provide localized treatment to the fingers or toes of a patient. It can be fitted (or not) with typical adhesives and release liner layers known in the bandaging arts, but need not have either adhesive nor release liner, and be provided instead in a roll for application to a wound or tissue area in the style of a gauze to be wrapped and held in place by conventional means. What is important to remember is the ratio of the two different metals.

Medical grade zinc metal flakes are used instead of generally rounded particulate zinc metals. This is because medical grade zinc metal flakes have shown that they have a greater surface area and thus are proven to have better galvanic current due to touch and electrochemistry. Typical medical grade zinc metal flakes will have dimensions of about 100 microns in diameter and are thin—having a thickness of about 0.25 millimeters. These medical grade zinc metal flakes applied with non-conductive water soluble carrier/binder binds the metals in suspension. This binder used is non-conductive in function. As the binder reduces in mass due to erosion during use, additional metallic materials contact the activation ingredient (saline) thereby allowing for additional ionic activity between the dissimilar metals.

Results show a 50% improvement over the original device of the '918 patent (whether non-touching/overlapping) with a sustained current of 25-30 micro-amps reading over the course of 3 days, with 25% amperage decay over 5 days consistent with expected mathematical exponential rate of decay. Expected decay of 15% formulation as manufactured is 11 days till sub 5 micro-amps. Galvanic skin responses as studied is roughly down to 0.05 microamps after initial burst. Lower than 0.05 micro-amps are not reliably measurable at this time. Measurement is point to point.

In a preferred embodiment, the metals deposition includes larger diameter circles (see FIG. 2) of deposited medical grade silver metal powder by a non-conductive water soluble carrier/binder, and smaller diameter circles of medical grade zinc metal flakes deposited in between the larger circles. 2 mm and 1 mm circles in an equal layout such that each silver dot is surrounded by at least 4 half dots of zinc to get the desired ratio of metals. All applied to the same side of said fabric. Spacing between metal formulations is approx. 1 mm in alternating rows. The diameters of the larger and smaller circles are not critical and can range from 1-6 mm and 0.5-3 mm, respectively. Preferably, the height of the deposition of the metals in the carrier is about 3 mm post curing. The patterns of circles can be very close but not to touch or overlap. The circles of the first metal type are spaced generally between about 1.5 to 2.5 mm apart so that intervening circles of the second metal type touch or overlap the circles of the first metal type. If the two metals touch or overlap a third printing process is involved to separate the two metals so as not to create a short in the current (see above). In other words, even an overlapping pattern, such as is shown in FIG. 2, does not involve dissimilar metals' actually touching, in the wound dressing as manufactured.

The carrier that the medical grade zinc metal flakes are deposited in can be any non-allergenic carrier/binder suitable for contact with skin or wound tissues. Preferably, a carrier/binder is fenestrated in the sense that it naturally or mechanically contains pores in it to provide access channels between the metals and extraneous fluids after the deposited metals and carrier have cured. Adequate liquid carrier/binder is used to suspend the metals into a viscous, thick ink-like substance which can be deposited onto the wound dressing pliable base and cured by drying or other curing method known in the art of printing or manufacturing. In addition, optionally carbon particles, carbon allotropes or other metals may be used to supplement the metal powders, particles or flakes to create conductive bridges either between metal types or even to deepen conductivity into the wound. In other words, conductive carbon allotropes bridges or other conductive materials may be used to interconnect the primary metals if necessary. When particulate carbon allotrope bridges are used, the metals of the present invention may not need to touch. The particles of a first metal such as silver are all touching within the deposited pattern held together by the carrier. The flakes of second metal all touch within the deposited pattern also. The first and second metals touch repeatedly as their respective patterns touch or overlap via a supplemental metal or flake creating the bridge.

Discussing now the operation of the wound dressing bandage illustrated, the treatment of a large variety of pathologies may be encouraged through the use of the present invention, including without limitation, infections, cuts, incisions (including surgical incisions), abrasions, lacerations, fractures, contusions, burns, and amputations. During use, the present wound dressing material is applied to a wound and the physiological fluids present in the wound area immediately contact the metal particles and immediately the electrolytic effect is to initiate a galvanic current in the area of the particles. A galvanic current of approximately 0.2 millivolts will form in the physiological fluids at the wound site. The galvanic current may range from 0.1 to 1.0 millivolts. The galvanic current performs as an antimicrobial against bacteria, viruses, fungi and empirically provides a profound healing effect to the tissues covered by the present wound dressing. Without intending to be bound by theory, the antimicrobial action occurs as follows: all bacteria, viruses, and fungi are negatively charged, so when the microbes are in the vicinity of, for example, silver and zinc and the resulting galvanic current, the negatively charged microbes migrate to and adhere to the silver particles 20. The action of the silver particles on the microbes is to interfere with the function of the Sulfhydryl (SH) groups of the microbes, and thus to interfere with the respiratory pathway of the organisms to kill the organisms. Along with their microcidal properties, the silver particles draw edema fluid from the wound area to decrease swelling which increases the capillary blood flow which promotes healing. Furthermore, ions from the other metal particles provide, in the case of zinc, therapeutic benefits including but not limited to, control of viruses and autolytic debridement of wounds and scar tissue. In the preferred embodiment, zinc is used because zinc is necessary for a wide variety of metabolic processes, including the synthesis as well as the degradation of nucleic acids, proteins, carbohydrates, and lipids. Zinc is also necessary for the synthesis of MMPs (metaloproteinase) which remodels the wound by degradation of nucleic acids, proteins, lipids, carbohydrates, and other breakdown products secondary to the cell destruction associated with wound healing. Zinc also adds a further bacteriocidal effect to the wound area. The high prevalence of zinc in mammal tissue speaks to its importance and role as a nutrient. Likewise, trace minerals from metals such as copper also affect tissue function. The direct application of the bimetallic wound dressing keeps the wound moist which aids healing. The galvanic current that results also decreases local pain in the manner of a “TeNS unit” (“transcutaneous electrical nerve stimulation unit”) which in turn decreases any limitation of movement, which also assists wound healing. Also, because the wound bed remains moist in the presence of the wound dressing, there is no pain or disruption of the healing process when the dressing is changed.

The production of a galvanic current by the present invention should be readily appreciated by one skilled in the art of electrochemistry. However, a brief overview is provided as follows: once the silver metal particles and the other metal particles are activated by an electrolyte-containing fluid, such as edema fluid, plasma, or blood which contains approximately 0.9% NaCl, ions are released. An electrical connection is formed between the particles and an electric current flows. Ions of the more active metal, the silver metal particles, which forms the anode, are transferred to the electrolyte to the less active metal, the other metal particles as the cathode. The movement of the ions creates an electrical galvanic current that produces a wound healing effect as discussed herein.

The presence of the silver anode, via the silver metal particles, and the zinc cathode, via the other zinc particles, creates a wet battery when moistened by wound fluids, thereby augmenting the current of injury by approximately one and a half microvolts which increases the deposition of the metal ions into the wound bed by iontophoresis. This deposition is in addition to the normal diffusion of the metal ions.

The controlled, sustained galvanic current created by the wound dressing of the present invention also enhances wound healing as follows: when the plasma or other physiological fluids at the edge of the wound come into contact with current produced by the present invention, the fluids become hypertonic. The hypertonicity of the fluids draws edema fluid from the wound edges. The removal of the edema from the wound edges not only keeps the wound bed moist but also decreases the pressure on the capillaries of the wound edge, thereby allowing increased blood flow into the wound, both of which enhance the healing process.

For the wound dressing of the present invention intended for eventual removal, the antimicrobial action will persist and makes it suitable to leave in place for up to ten days. If necessary, the wound dressing may even be rinsed with water and reapplied in settings in which a substitute wound bandage is not available, such as in combat or other extremis. Additionally, because of the inherent antimicrobial character of the present invention, the present wound dressing can even be used in emergencies and dire circumstances as an emergency water purification filter. In an emergency, the wound dressing may be doubled over and used as a water filter to separate out bacteria and solid contaminants. It is believed that the exposure of the microbes to the electrical current formed by the silver and other metal particles kills and/or removes enough microbial contamination from the water to make nonpotable water generally potable. While there may be limitations on the ability of the present invention to purify water under some conditions, this disclosure of gross decontamination action should be understood as an emergency field measure when no other water purification equipment or chemicals are available. Such conditions would foreseeably arise under the same circumstances as would the need for wound dressing materials, namely, armed services and terrorist abatement deployments of all kinds

Prior to use of the wound dressing of the present invention, it may be desirable to activate it by delivering a suitable liquid such as water, saline solution and solution or lactated saline solution (Ringer's Solution) to the surface containing the deposited metals. In some cases, it may be desirable to provide other metals via the liquid. The activating liquid can also comprise drugs or agents for therapeutic effects or to retain moisture, or to provide nutrition directly to tissue, such as fetal calf serum.

An important advantage of all embodiments of the present invention is that they provide sustained release therapeutic and/or antibacterial, anti-fungal and antiviral properties without the need for an external power source. This reduces the cost of devices, simplifies uses and enhances reliability. It is understood that the specification and drawings are illustrative of, but do not limit, the present invention, and other embodiments and variations are within the spirit and scope of the present invention.

The present invention has been described with reference to the preferred embodiments. Obvious modifications, combinations or alterations will occur to others upon reading the preceding detailed description. It is intended that the invention be construed as including all such modifications, combinations and alterations insofar as they come within the scope of the appended claims or the equivalents thereof

Throughout the instant technology, galvanic voltage max is elementally calculated to be 1.558 volts. Current design runs at 0.6 volts equaling 38% voltage efficiency at perfection. Assuming 10% material and measurement error, Current design equals 43% efficiency. The following steps bring that closer to perfection: 1. Ideal ratio of metal in formulation is 0.865 Silver to 1.0 Zinc; therefore, the ideal formulation would be 15% silver/85% binder and 18% zinc/82% binder in formulation form. 2. Aluminum could replace Zinc in formulation for a 30% improvement in voltage.(Greater if oxidation is controlled). 3. Binder can be made conductive for a calculated 86% improvement in current potential. 4. An additional (Third) ionic compound/element could be added to the layout to create an additional harmonic waveguide. (This can be tailored.) 5. Said Third compound/element could allow structure to act as a harmonic oscillator allowing for structured/desired measureable response(s), i.e.; carbon allotropes/graphene. 6. Implanting of metal/ionic formulations in thicker materials (sponges) allows for closer spacing which leads to greater current flow. 7. Current flow can work backwards allowing for measured environmental response(s). Note studies show that medical grade silver metal powder and medical grade zinc metal flakes can touch and or overlap with same results if a third printing process is is added to separate the two metals from touching and creating a short in the battery.

In view of all of the above, the present Advanced Electrolytic Device-Bimetallic Wound Dressing provides galvanic current and has antimicrobial properties as follows. The embodiment of the added studies of the invention in which medical grade silver metal powder has been deposited by a non-conductive water soluble carrier/binder on a pliable dressing base material medical grade 100% polyester fabric material has shown a 50% improvement over the original device with a sustained current of 25-30 micro-amps reading over the course of 3 days and only a 25% amperage decay over 5 days consistent with expected mathematical exponential rate of decay. Expected decay of 15% formulation as manufactured is 11 days till sub 5 micro-amps. Galvanic skin responses as studied is roughly down to 0.05 micro-amps after initial burst. Measurement is point to point. Other materials can be used such as non-allergenic gauze or open-celled foam or other pliable materials compatible to non-irritation of the skin or wound bed, in a pattern of repeating circles whereas silver powder are deposited by same non-conductive water soluble carrier/binder on same stated 100% polyester medical grade material fabric in a coordinated pattern of 4 half dots of Zinc to get the desired ratio of metals.

In addition the silver mixture can be applied on one side of the fabric/permeable surface and the zinc mixture can be applied on the other side of the fabric/permeable surface; as long as the material is water permeable, this latter layout works as well as the single surface area application without weakening the galvanic current needed for wound healing.

Although the invention has been described with particularity above, with reference to specific materials and methods, the invention is only to be limited insofar as is set forth in the accompanying claims.

1. A wound dressing, comprising a base material; a surface suitable for contacting a wound, and a pattern of deposited medical grade metal powder and flakes on said surface wherein at least two types of metals are present, comprising a first metal having an electrochemical potential and a second metal having a different electrochemical potential from said first metal, said metals being deposited and adhered to said surface in a non-conductive water soluble carrier/binder to create metal formulations deposited in a non-touching pattern, further wherein said dressing comprises a pliable base material and said metal formulations comprise at least two metals which, upon nearly touching, generate an efficient and sustainable galvanic current and wherein said layout is such that the ratio of metallic formulations is as close to one-to-one silver to zinc as possible by screening 2 mm and 1 mm circles in an equal layout such that each silver dot is surrounded by at least 4 half dots of zinc to achieve the desired ratio of metals, and all dots are applied to the same side of said fabric with spacing between said metal formulations' being approximately 1 mm in alternating rows.

2. The wound dressing according to claim 1, wherein said first metal is medical grade silver metal powder and said second metal is medical grade zinc metal flakes.

3. The wound dressing according to claim 1, wherein said first metal is medical grade silver metal powder or silver salts or oxides and said second metal is aluminum, cobalt, copper, gold, iron, magnesium, platinum, titanium or zinc, and/or suitable salts and oxides thereof

4. The wound dressing according to claim 1, wherein said metals are deposited in circles as part of said pattern.

5. The wound dressing according to claim 4, wherein said deposited metals have a deposition height of no higher than 3 mm.

6. The wound dressing according to claim 5 wherein said deposited metals are metal powder and flakes having approximately 100 micron diameters and thicknesses of approximately 0.25 mm.

7. The wound dressing according to claim 6 wherein said carrier/binder is a non-conductive binder.

8. The wound dressing according to claim 6 wherein said carrier/binder is a fenestrated non-conductive binder.
